(19) United States
(12) Patent Application Publication
(10) Pub. No.: US 2023/0320569 A1
(43) Pub. Date: Oct. 12, 2023

(54) Endoscopic Attachment, Cap for Endoscope and Endoscopic System

(71) Applicants: Kenneth F. BINMOELLER, Rancho Santa Fe, CA (US); MICRO-TECH (NANJING) CO., LTD., Nanjing (CN)

(72) Inventors: Kenneth F. BINMOELLER, Rancho Santa Fe, CA (US); Scott HAACK, Chardon, OH (US); Lei XIE, Nanjing (CN)

(21) Appl. No.: 18/208,651

(22) Filed: Jun. 12, 2023

Related U.S. Application Data
(63) Continuation-in-part of application No. PCT/CN2021/137253, filed on Dec. 10, 2021.
(60) Provisional application No. 63/125,389, filed on Dec. 14, 2020.

(51) Int. Cl. A61B 1/00; A61B 1/018; A61B 17/00

(52) U.S. Cl. CPC A61B 1/00087; A61B 1/00094; A61B 1/018; A61B 17/00234; A61B 1/00091; A61B 2017/00296

(57) ABSTRACT

An endoscopic attachment and an endoscopic system are provided. The endoscopic attachment includes a proximal portion configured to be coupled to an insertion tube of the endoscope; and a distal portion coupled to the proximal portion and extending forwardly from the proximal portion to a forward edge. The distal portion includes a first axial segment that extends from the proximal portion axially to the forward edge by a first axial length, defines a passage, and extends circumferentially entirely around an axis of the passage; and a second axial segment that extends a second axial length from the first axial segment to the forward edge, and extends circumferentially around the axis of the proximal portion less than 360 degrees over at least part of the second axial length. The endoscopic attachment allows for tissues to be cut more efficiently and/or effectively during ESD, POEM and other procedures.

The disclosure is a Continuation-in-Part of the PCT International Application No. PCT/CN2021/137253, entitled “Endoscopic Accessory for Gastrointestinal Endoscopy”, filed on Dec. 10, 2021; which application, pursuant to 35 U.S.C. § 119 (e), claims priority to the filing date of U.S. Provisional Patent Application Ser. No. 63/125,389, filed Dec. 14, 2020; the disclosures of which applications are incorporated herein by reference.

### TECHNICAL FIELD

The disclosure relates to the technical field of endoscopy, and in particular to a cap attachment for an endoscope and an endoscopic system.

### BACKGROUND

In recent years, gastrointestinal diseases such as mucosal tumors and achalasia have been treated by peroral flexible endoscopy. Take achalasia as an example, which is a condition of the esophagus in which the lower esophageal sphincter does not operate properly, hindering passage of food to the stomach. Achalasia may result in difficulty swallowing, weight loss, and regurgitation, among other symptoms.

Achalasia may be treated by peroral endoscopic myotomy, referred to herein after as “POEM”, wherein the muscle fibers of the lower esophageal sphincter are cut. To perform POEM, an endoscope is inserted through an incision made in the mucosa of the esophagus into the submucosal space between the mucosa and the muscularis propria. A tunnel is then created by submucosal dissection with a catheter-mounted knife, referred to hereinafter as “knife”, inserted through the endoscope working channel until the lower esophageal sphincter is reached. Myotomy of the lower esophageal sphincter is then performed by cutting the muscle fibers with the knife.

Mucosal tumors may be treated by endoscopic submucosal dissection, which is referred to herein after as “ESD”. ESD entails making an incision in the mucosa outside of the boundaries of the tumor and entering the submucosal space between the mucosa and the muscularis propria to dissect the tumor-bearing mucosa free from the underlying muscularis propria with a knife inserted through the endoscope.

### SUMMARY

An aspect of an embodiment of the disclosure provides an endoscopic attachment including a proximal portion and a distal portion, wherein the proximal portion is configured to be coupled to an insertion tube of the endoscope, the distal portion is coupled to the proximal portion and extends forwardly from the proximal portion to a forward edge; wherein the distal portion includes a first axial segment and a second axial segment, wherein the first axial segment extends from the proximal portion axially to the forward edge by a first axial length, and defines a passage and extends circumferentially entirely around an axis of the passage; the second axial segment extends a second axial length from the first axial segment to the forward edge, and over at least part of the second axial length, the second axial segment extends circumferentially around the axis of the proximal portion less than 360 degrees.

In a possible embodiment, over the second axial length, the second axial segment extends circumferentially about the axis less than or equal to 180 degrees on average.

In a possible embodiment, a lateral notch which transverses to said axis and extends through the passage is formed over the forward edge.

In a possible embodiment, an open surgical space is enclosed between the forward edges, and the surgical space is configured to accommodate a knife extending from the distal end of the insertion tube to the distal portion;

wherein the tip of the knife is axially flush with or protruding from the lateral notch.

In a possible embodiment, the forward edge is axially recessed to form a lateral notch relative to a plane extending laterally across the passage and intersecting both a short side of the forward edge and the second axial segment, respectively, the short side being the portion of the forward edge at a first axial length in the first axial segment.

In a possible embodiment, the plane intersects the long side of the forward edge, the long side being the portion of the forward edge at the second axial length in the second axial segment.

In a possible embodiment, the forward edge extends a first axial length over a first circumferential span of 0 to 180 degrees about the axis.

In a possible embodiment, the forward edge extends a first axial length over a first circumferential span of 30° to 150° around the axis.

In a possible embodiment, the forward edge extends a second axial length over a second circumferential span of 0 to 180 degrees about the axis.

In a possible embodiment, the first axial length is 2 mm to 15 mm.

In a possible embodiment, the second axial length is 2 mm to 15 mm.

In a possible embodiment, the second axial length is 2 mm to 5 mm.

In a possible embodiment, the second axial segment transitions moving axially from the circular cross-section shape of the first axial segment to having a flattened side.

In a possible embodiment, the endoscopic attachment further includes one or more drain holes.

In a possible embodiment, at least one of the drain holes is located in the first axial segment, and the distance thereof from the proximal portion is smaller than the first axial length;

alternatively, at least one of the drain holes is located at a junction of the proximal portion and the first axial segment;

alternatively, at least one of the drain holes is located on the second axial segment;

alternatively, at least two of the drain holes are located on the second axial segment, and at least two of the drain holes are circumferentially aligned by the extension sides formed by the second axial segment

In a possible embodiment, an adhesive tape with a non-adhesive removable coating is secured to a rearward edge, and during mounting on the insertion tube, the adhesive tape is folded backward over the rearward edge, wherein the rearward edge is an edge of an end of the proximal portion away from the distal portion.

In a possible embodiment, the adhesive tape is secured to a surface of the insertion tube by peeling off the non-adhesive coating.

In a possible embodiment, the circumferential sidewall of the second axial segment extends in a direction coincident with the axis.

In a possible embodiment, the lateral notch over the forward edge is provided between the short side of the forward edge and the axis; the short side is a portion of the forward edge at a first axial length in the first axial segment.

In a possible embodiment, at least a portion of said second axial segment is inclined toward said axis in a direction away from said first axial segment, and an end of the at least portion of the second axial segment is located at the second axial length of the second axial segment.

In a possible embodiment, the second axial segment is gradually inclined from said first axial segment towards said axis throughout the second axial length.

In a possible embodiment, the second axial segment and the short side of the forward edge are located on either side of a first section plane, respectively; wherein the first section plane is a longitudinal section passing through the axis, and the short side is the portion of the forward edge at the first axial length in the first axial segment.

In a possible embodiment, circumferentially opposite side edges of the second axial segment are located on the first section plane.

In a possible embodiment, the lowest portion of the lateral notch on the forward edge is flush with or below the first section plane.

In a possible embodiment, a projection of the circumferential sidewall of the second axial segment on the distal end of the insertion tube is arranged in a staggered manner with respect to the optical system at the distal end of the insertion tube.

In a possible embodiment, an avoidance port is formed at an end of the second axial segment away from the first axial segment, and an axial projection of the optical system on the second axial segment is located in the avoidance port.

In a possible embodiment, an end of the second axial segment facing away from the first axial segment meets with the axis.

In a possible embodiment, the circumferential sidewall of the second axial segment is inclined toward the axis at an angle of 15°-60°.

Another aspect of the disclosure discloses an endoscopic system, including:

an endoscope having an insertion tube and a knife extendable from the distal end of the insertion tube; and

the endoscopic attachment as above, the endoscopic attachment is coupled to the insertion tube at the distal end.

In a possible embodiment, the axis is located radially between the knife and a portion of the second axial segment extending the second axial length to the forward edge.

Yet another aspect of the disclosure discloses an endoscopic system, including: a coupling portion configured to be coupled to an insertion tube of an endoscope having a knife extendable from the insertion tube; and a shroud portion extending axially from the coupling portion to a forward edge, the shroud portion defining a passage having an axis and a lateral notch extending through the channel transverse to the axis.

In a possible embodiment, the lateral notch is an axially forward region of the forward edge and an axially rearward region of a plane which extends through the passage and intersects the forward edge on the opposite sides of the forward edge.

In a possible embodiment, the forward edge is recessed with respect to the plane.

In a possible embodiment, the coupling portion is configured to be coupled to the insertion tube in a direction such that the knife may extend into the lateral notch.

In a possible embodiment, the shroud portion extends circumferentially about said axis less than or equal to 180 degrees on average over an axial length including the forward edge.

In the endoscopic attachment and the endoscopic system provided by the embodiments of the disclosure, by setting the endoscopic attachment to include a first axial segment and a second axial segment and setting the first axial segment to incline in the axial direction of the endoscope, the second axial segment is set to extend forward from the first axial segment, so that when a gastrointestinal endoscopic surgery (such as POEM or ESD) is performed, after the mucosal layer is cut by the knife of the endoscopic system of the embodiment of the disclosure, the second axial segment lift the mucosal layer, so that the entire second axial segment enters the submucosa between the mucosal layer and the muscularis propria gradually, and during the endoscope carrying cap continues to extend into the submucosa, tissue such as the submucosa is suspended tensely over the forward edges of the first and second axial segments, while also supporting the tissue away from the distal end of the endoscope, thereby increasing the field of view of the endoscope and increasing the surgical operation space at the front end of the second axial segment. In addition, the first axial segment extends the first axial length forward in the axial direction to increase the distance between the second axial segment and the distal portion of the endoscope, the first axial segment will horizontally support part of the tissue in the rearward region of the second axial segment, so that the blockage of the field of view of the optical system, such as the camera, by the tissue supported on the outer surfaces of the first axial segment and the second axial segment can be improved or eliminated, so that the surgical process can be carried out more quickly and accurately.

In addition, the cap of the endoscope provided by the embodiment of the disclosure is provided with a passage on the shroud portion and a lateral notch extending through the channel transverse to the axis, so that when a gastrointestinal endoscopic surgery (such as POEM or ESD) is carried out, the tissue is supported away from the distal end of the insertion tube by the forward edge of the shroud portion, thereby increasing the field of view of the endoscope and increasing the surgical operation space at the front end of the shroud portion. In addition, when the tissue is supported on the forward edge, it may sink in the lateral notch, so that the knife does not need to extend a long distance and only needs to be exposed to the lateral notch to contact and cut the tissue, shortening the extension length of the knife and improving the operability of the knife. In addition, when the knife is in operation, it can be exposed to the lateral notch to a greater extent, which further improves the operating field of view of the endoscopic operation, and makes the operation of the entire endoscope more reliable, accurate and efficient.

### BRIEF DESCRIPTION OF THE DRAWINGS

The disclosure is best understood from the following detailed description when read in conjunction with the accompanying drawings. It is emphasized that, according to common practice, the various features of the drawings are not to scale. On the contrary, the dimensions of the various features are arbitrarily expanded or reduced for clarity.

FIG. 1 is a schematic view of an endoscope inserted into the submucosa during a peroral endoscopic myotomy procedure provided by an embodiment of the disclosure;

FIG. 2 is a cross-sectional view taken along line 2-2 in FIG. 1;

FIG. 3a is a first structural schematic view of an endoscopic system provided by an embodiment of the disclosure;

FIG. 3b is a schematic structural diagram of an endoscopic system with adhesive tape provided by an embodiment of the present disclosure;

FIG. 4 is a schematic structural diagram of an endoscope provided by an embodiment of the present disclosure;

FIG. 5 is a schematic structural view of the cap in FIG. 3a;

FIG. 6 is a left elevation view of FIG. 5;

FIG. 7 is a top view of FIG. 5;

FIG. 8 is a left elevation view of FIG. 3a;

FIG. 9 is a top view of FIG. 3a;

FIG. 10 is a front view of FIG. 3a;

FIG. 11 is a front view of FIG. 3a with various dimensions labeled;

FIG. 12 is a schematic structural view of the endoscopic system in FIG. 3a during a gastrointestinal surgery;

FIG. 13 is a second schematic structural view of a cap provided by an embodiment of the present disclosure;

FIG. 14 is a left elevation view of FIG. 13;

FIG. 15 is a top view of FIG. 13;

FIG. 16 is a third schematic structural view of the endoscopic system provided by an embodiment of the present disclosure;

FIG. 17 is a left elevation view of the endoscopic system in FIG. 16;

FIG. 18 is a fourth schematic structural view of the endoscopic system provided by an embodiment of the present disclosure;

FIG. 19 is a left elevation view of FIG. 18;

FIG. 20 is a front view of FIG. 18;

FIG. 21 is a schematic structural view of the cap in FIG. 18;

FIG. 22 is a left elevation view of FIG. 21;

FIG. 23 is a bottom view of FIG. 21;

FIG. 24 is a first schematic structural view of the endoscopic system in FIG. 18 during a gastrointestinal surgery;

FIG. 25 is a second schematic structural view of the endoscopic system in FIG. 18 during a gastrointestinal surgery;

FIG. 26 is a fifth schematic structural view of a cap provided by an embodiment of the present disclosure;

FIG. 27 is a sixth schematic structural view of a cap provided by an embodiment of the present disclosure;

FIG. 28 is a top view of FIG. 27.

### DETAILED DESCRIPTION

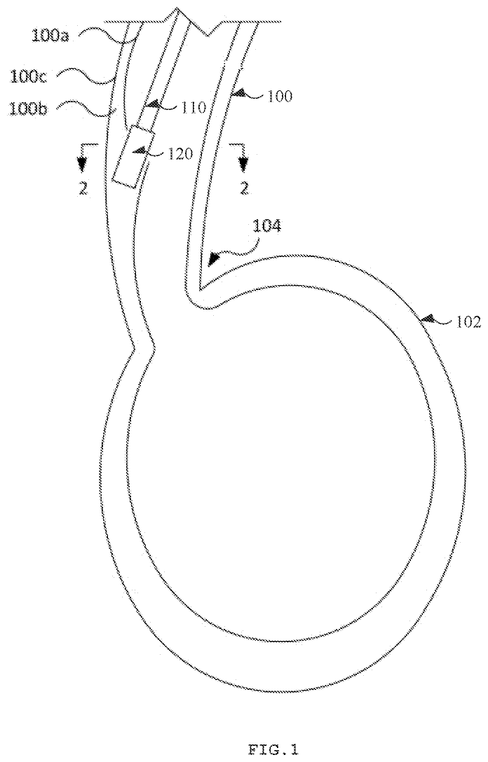

FIG. 1 is a schematic view of an endoscope inserted into the submucosa during a peroral endoscopic myotomy procedure provided by an embodiment of the disclosure, and FIG. 2 is a cross-sectional view taken along line 2-2 in FIG. 1. Referring to FIGS. 1 and 2, an embodiment of the disclosure illustrates an endoscopic system, components and surgical procedures for performing POEM. Although disclosed with specific reference to POEM, it should be readily understood that the endoscopic system and the endoscopic attachment described in the disclosure may also be used in other endoscopic surgical procedures, such as endoscopic submucosal dissection (ESD for short) or other endoscopic gastrointestinal procedures. The endoscopic system includes a knife, one or more water sources, a camera, an endoscope, and ENDOSCOPIC ATTACHEMENT. The ENDOSCOPIC ATTACHEMENT, which may also be referred to herein as a cap, includes an extended side that may allow for more efficient and/or effective cutting of tissue by the endoscopic system, such as the submucosa and muscle fibers of the lower esophageal sphincter, during POEM or other procedures.

Referring to FIGS. 1 and 2, a schematic of the esophagus 100 is shown with an endoscope 110 inserted therein during a POEM procedure. The esophagus 100 extends from the pharynx (not labeled) to the stomach 102. The esophageal sphincter 104 is located at the bottom of the esophagus 100 adjacent the stomach 102. As shown in the cross-section of FIG. 2, the esophagus 100 generally includes the mucosa 100a, the submucosa 100b, and the muscularis propria 100c. The mucosa 100a is the innermost layer of the esophagus 100. The submucosa 100b is an intermediate layer of tissue positioned between the mucosa 100a to the muscularis propria 100c. The muscularis propria 100c, which may also be referred to as the “MP,” is positioned outward of and adjacent to the submucosa 100b. The muscularis propria 100c is the muscle that provides motility to move food downward through the esophagus 100 to the stomach 102. The lower esophageal sphincter 104 includes, as referenced above, muscle tissue and is located at a lower end of the esophagus 100 adjacent the stomach 102.

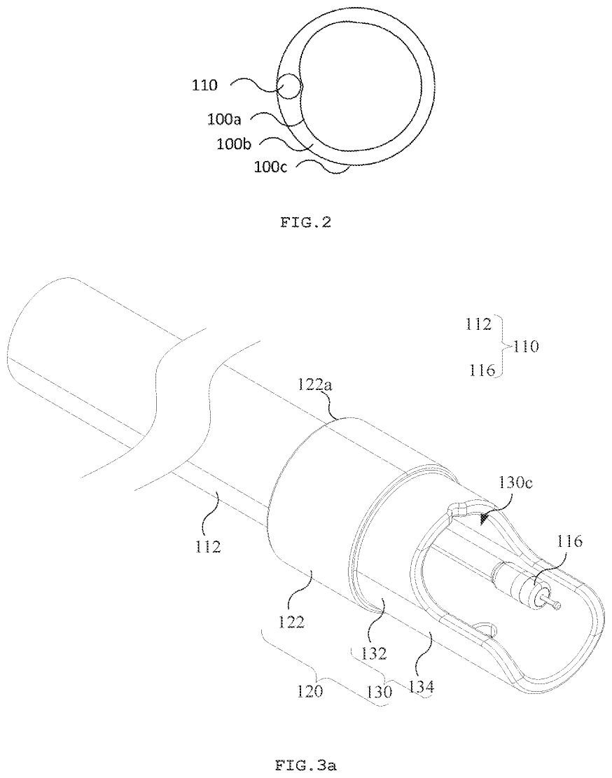

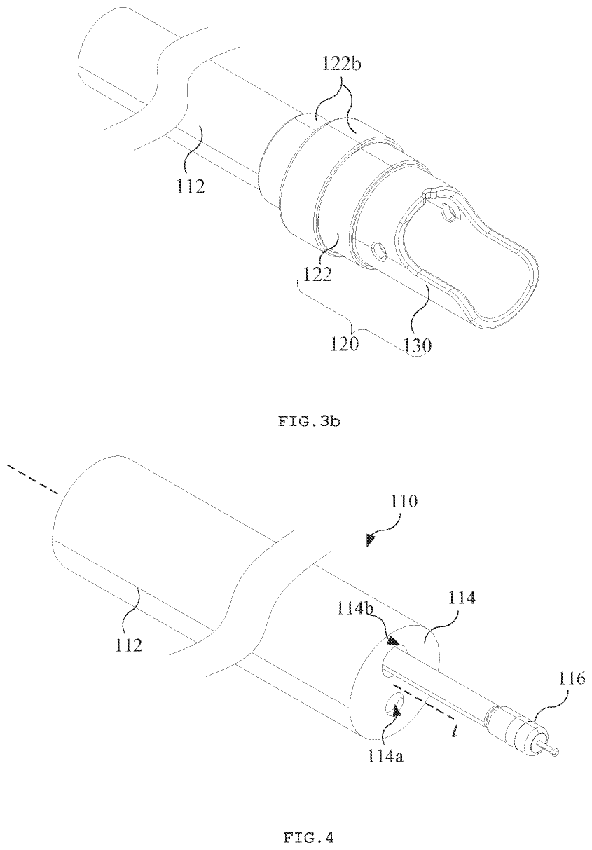

FIG. 3a is a first structural schematic view of an endoscopic system provided by an embodiment of the disclosure, FIG. 3b is a schematic structural diagram of an endoscopic system with adhesive tape provided by an embodiment of the present disclosure, and FIG. 4 is a schematic structural diagram of an endoscope provided by an embodiment of the present disclosure. Referring to FIGS. 3a and 4, the endoscope 110 in FIG. 3a generally includes an insertion tube 112 having a distal end 114. The insertion tube 112 may, as shown in FIG. 4, be generally cylindrical and terminate at the distal end 114. The insertion tube 112 includes one or more channels (not illustrated) extending therethrough to one or more nozzles 114a in the distal end 114 that supply water (including other fluid solutions, such as saline) and “air” (including other gases, such as carbon dioxide).

The insertion tube 112 also includes dedicated channels (not illustrated) extending therethrough to outlets 114b in the distal end 114, for supply of gas, suction and insertion of instruments. The outlet 114b provides an outlet for an instrument, such as a knife 116, to be extended from the distal end 114 of the endoscope 110 to engage tissue of the patient. When an instrument is inserted into/through the outlet 114b, the inserted instrument will not be positioned off-center from the axis of the outlet 114b. As shown in FIG. 4, the outlet 114b may be biased toward one side of the distal end 114, for example, being positioned off-center from an axis l of the distal end 114. As a result, the knife 116 is similarly biased toward one side of the distal end 114 of the endoscope 110, such as being off-center from the axis l. As shown, the outlet 114b, as well as the knife 116, may be positioned radially between the axis l and an outer periphery of the distal end 114 of the insertion tube 112.

In practice, the distal end 114 of the endoscope 110 may also include one or more light-guide lenses (not shown) and an object lens (not shown). The one or more light-guide lenses emit light from a light source to provide illumination forward of the distal end 114 of the insertion tube 112. For example, the insertion tube 112 may include optical fibers (e.g., glass fibers) or other means extending therethrough (not illustrated) that transfer light from the light source to the light-guide lenses for illumination purposes. The insertion tube may further include other optical fibers or other optical transmission means extending therethrough (not illustrated) that transfer light from the object lens to the camera for imaging purposes.

Endoscope 110 also accommodates a knife 116 inserted through the outlet 114b, such as an electrosurgical knife specifically configured for endoscopic submucosal dissection. The knife 116 includes a tip having an electrode and which may be any suitable shape (e.g., spherical, triangular, hook shaped), be insulated or non-insulated, and/or may provide water injection. When configured for water injection, the knife 116 may be an additional source of water to the nozzle 114a.

Though not shown, the endoscope 110, or endoscopic system that includes the endoscope 110, may also be considered to include the various fluid and light sources described above (e.g., water, air, suction, and/or light), the camera, the instrument (e.g., the knife 116), and/or controls for operation thereof.

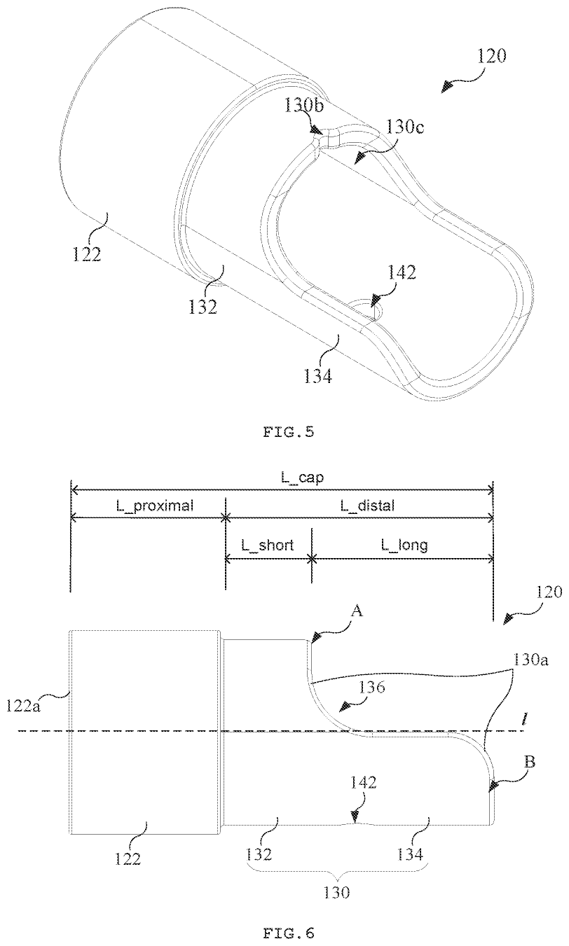

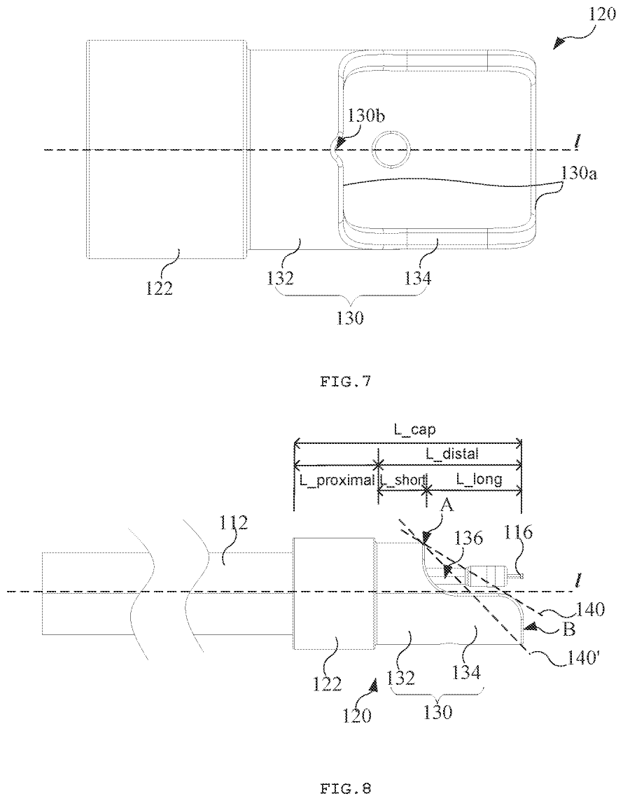

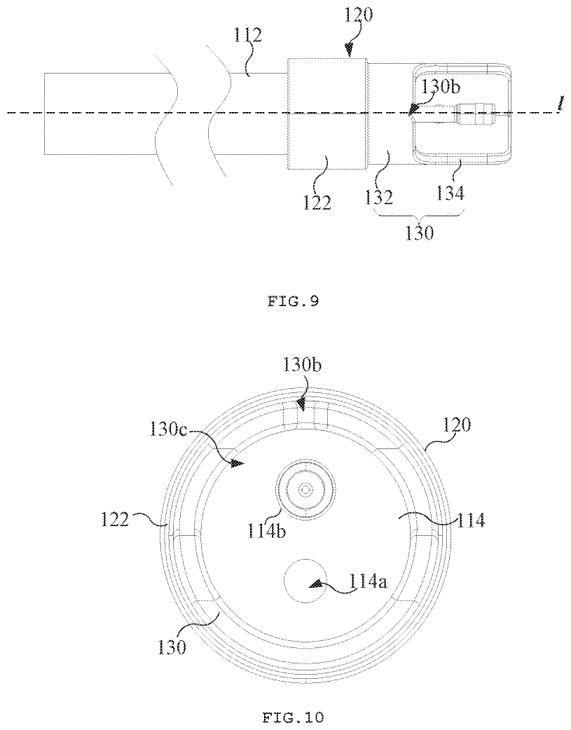

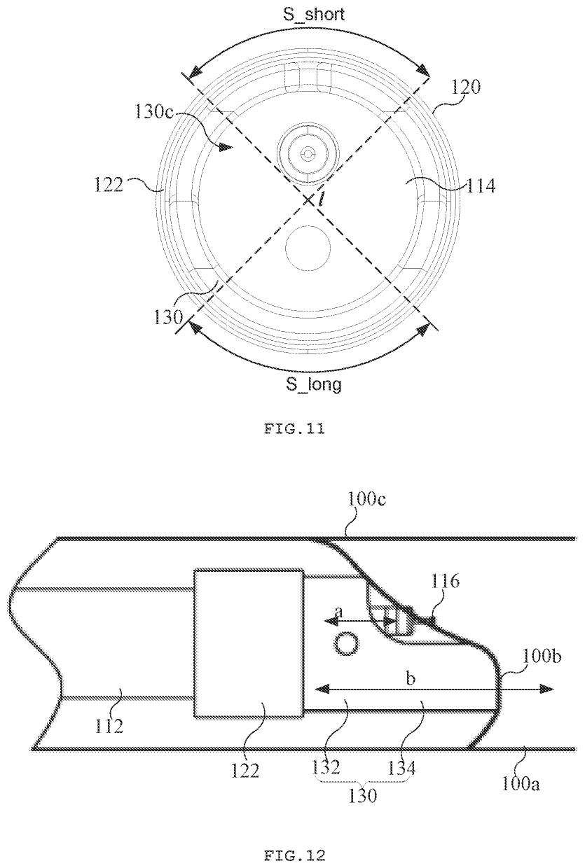

FIG. 5 is a schematic structural view of the cap in FIG. 3a, FIG. 6 is the left elevation view of FIG. 5, FIG. 7 is a top view of FIG. 5, FIG. 8 is a left elevation view of FIG. 3a, FIG. 9 is a top view of FIG. 3a, FIG. 10 is a front view of FIG. 3a, FIG. 11 is a front view of FIG. 3a with various dimensions, FIG. 12 is a schematic structural view of the endoscopic system in FIG. 3a performing gastrointestinal surgery. Referring to FIGS. 5 to 12, the endoscopic system of the embodiment of the disclosure further includes a cap 120. The cap 120 is an attachment that couples to the distal end 114 of the endoscope 110 and is configured to press or otherwise engage tissue in manners to facilitate viewing and cutting thereof. As referenced above and discussed further below, the cap 120 includes an extended side (e.g., extension, axially-extending protrusion, flange, or tip). The cap 120 may also be referred to as an attachment, a distal attachment, or a hood.

Referring to FIGS. 5 to 7, the cap 120 is tubular and generally includes a proximal portion 122 and a distal portion 130 coupled to the proximal portion 122 and extending forward from the proximal portion 122. The tubular cap 120 allows water, air, tissue, light and/or instruments such as the knife 116 to pass therethrough to and/or away from distal end 114 of insertion tube 112. The cap 120 extends between a rearward edge 122a (where the proximal portion 122 terminates) and a forward edge 130a (where the distal portion 130 terminates).

As shown in FIGS. 6 and 7, the cap 120 may have a length L_cap measured axially (i.e., generally parallel with the axis l of the insertion tube 112 and/or the cap 120) from the rearmost portion of the rearward edge 122a to the forwardmost portion of the forward edge 130a. The length L_cap of the cap 120 may be approximately 10 mm to 40 mm, such as 15 mm to 30 mm (e.g., 15 mm to 20 mm, 20 mm to 25 mm, or 25 mm to 30 mm), or another suitable distance. As discussed in further detail below, the axial length L_cap of the cap 120 includes, and may be equal to a sum of, a length L_proximal of the proximal portion 122 and a length L_distal of the distal portion 130.

Referring to FIGS. 3, 8 and 9, the proximal portion 122 is configured to couple to the distal end 114 of the insertion tube 112. For example, the proximal portion 122 may be generally tubular and configured to receive the distal end 114 of the insertion tube 112 therein. The proximal portion 122 of the cap 120 may connect with the insertion tube 112 of the endoscope 110 by at least one of a friction fit (e.g., inner surface of the proximal portion 122 of the cap 120 frictionally engages and/or compresses the distal end 114 of the endoscope 110) or an adhesive tape.

Referring to FIG. 3b, in some embodiments, the rearward edge 122a of cap 120 comprises tape 122b with a removable non-adhesive coating. During installation of the cap 120 onto the insertion tube 112, the tape 122b is folded back onto the rearward edge 122a. The cap 120 is secured to the surface of insertion tube 112 by peeling off the non-adhesive coating to expose the adhesive surface of the tape 122b. The proximal portion 122 may also enable the cap 120 to couple to the endoscope 110 in one or more orientations as may be determined by the user, for example, to orient the extended side of the cap 120 relative to the knife 116 (e.g., being nearest or furthest therefrom). The proximal portion 122 may also be referred to as the coupling portion. The desired positioning of the extended side of the cap 120 relative to the knife 116 is determined by a steep notch or printed mark at the midpoint of the extended side of the cap 120, which is visible externally or internally in endoscopic imaging.

The proximal portion 122 being generally tubular includes an outer surface and an inner surface with a thickness extending therebetween. In some examples, the outer surface and the inner surface are cylindrical and coaxial, such that the thickness of the sidewall of proximal portion 122 is constant extending both circumferentially around the proximal portion 122 and axially therealong. The inner surface of the proximal portion 122 has a diameter that allows the distal end 114 of the endoscope 110 to be received therein, for example, of 6 mm to 20 mm (e.g., 7 mm to 12 mm, such as approximately 9 mm), depending on the insertion tube 112 of the endoscope 110. While discussed and illustrated as being cylindrical, the proximal portion 122 (e.g., the outer surface and/or the outer surface) may have other shapes, to facilitate coupling to other endoscopes 110 and/or coupling mechanisms.

Referring to FIGS. 6 and 8, the length L_proximal of the proximal portion 122 extends axially from the rearward edge 122a of the cap 120 to the distal end 114 of the endoscope 110 (e.g., that distance coupling to and/or overlapping the insertion tube 112 of the endoscope 110). The length L_proximal of the proximal portion 122 may be between approximately 4 and 15 mm, such as 6 mm to 10 mm (e.g., approximately 8 mm).

The distal portion 130 forms the extended side of the cap 120. Referring to FIG. 11, in use, the distal portion 130 of the cap 120 engages tissue (e.g., of the submucosa 100b) and holds the tissue away from the distal end 114 of the endoscope 110. This provides a field of view to the camera, while also allowing manipulation of the knife 116 for engaging and cutting the tissue (e.g., of the submucosa 100b and/or the propria muscularis 100c, including the lower esophageal sphincter 104, during POEM).

Referring to FIG. 3a, the distal portion 130 is generally tubular and defines a passage 130c through which the water, air, suction, and/or light pass to and/or from the distal end 114 of the endoscope 110. The knife 116 is also extended and retracted through the passage 130c for cutting tissue. The distal portion 130 may also be referred to as a hood portion.

Referring to FIG. 6, the length L_distal of the distal portion 130 extends axially from the proximal portion 122 (e.g., from the distal end 114 of the endoscope 110) to a forward edge 130a of the distal portion 130 axially furthest from the endoscope 110. The axial length L_distal of the distal portion 130 may have the axial length L_distal, for example, of approximately 5 mm to 25 mm, such as 10 mm to 20 mm (e.g., approximately 15 mm) or such as 5 mm to 15 mm (e.g., approximately 8 mm to 10 mm). The axial length L_distal may include and be equal to a sum of an axial length L_short of the first axial segment 132 and an axial length L_long of the second axial segment 134, as discussed in further detail below. The length L_short may also be referred to as the first axial length, and the length L_long may also be referred to as the second axial length.

Referring to FIGS. 6 and 7, the distal portion 130 of the cap 120 forms the extended side by extending different distances axially forward from the proximal portion 122 (e.g., from the distal end 114 of the endoscope 110) to the forward edge 130a. More particularly, as discussed in further detail below, the distal portion 130 includes a first axial segment 132 and a second axial segment 134 that extends further axially from the proximal portion 122 to the forward edge 130a than the first axial segment 132, so as to form the extended side.

Wherein, the first axial segment 132 extends from the proximal portion 122 axially forward to the edge 130a for a first axial length, and the second axial segment 134 extends from the first axial segment 132 to the forward edge 130a for a second axial length. An open surgical space is enclosed between the forward edges 130a of the first axial segment 132 and the second axial segment 134, and the knife 116 extending from the distal end 114 of the insertion tube 112 into the distal portion extends into the surgical space.

During gastrointestinal endoscopy or surgery, such as with POEM, the forward edges 130a of the first axial segment 132 and the second axial segment 134 engages (or abuts) the tissue such as submucosa 100b, such that the submucosa 100b spans the surgical space. Either the muscularis propria 100c or the mucosal layer 100a is supported on the external surface of the long side B of the forward edge 130a and conversely either the mucosal layer 100a or the muscularis propria 100c is supported on the outer side surface of the short side A of the forward edge 130a. Wherein, the short side A is the portion of the forward edge 130a at the first axial length in the first axial segment 132. The long side B is the portion of the forward edge 130a at the second axial length in second axial segment 134.

Next, the knife 116 is manipulated in direction a (referring to FIG. 12) so that the knife 116 protrudes forward to contact and cut tissues. Next, the whole endoscope 110 is manipulated to move in the direction b, so that the knife 116 continues to protrude forward to contact and cut a new tissue. The above-mentioned structural arrangement of the first axial segment 132 and the second axial segment 134, on the one hand, maintains a certain distance between the tissue and the distal end 114 of the endoscope 110, providing a wider field of view of the submucosal space for an optical system such as a camera, and on the other hand, by means of the abutting support of the forward edge 130a, a traction effect on the submucosal tissue to be cut to make it easier for the knife 116 to cut the tissue.

The first axial segment 132 of the distal portion 130 of the cap 120 includes that point or portion of the forward edge 130a that, measured in the axial direction (e.g., parallel with the axis l), is nearest to the proximal portion 122 and/or the distal end 114 (see FIGS. 6 and 7) of the endoscope 110.

The first axial segment 132 of the cap 120 may extend substantially continuously (e.g., entirely) around the axis l to form the passage 130c. The first axial segment 132 of the cap 120 thereby defines a volume of the cap 120 (e.g., of the passage 130c), which may be substantially cylindrical or have another suitable shape.

It can be understood that, the extension direction of the circumferential side wall of the first axial segment 132 is consistent with the extension direction of the axis l, which ensures that the first axial segment 132 will not block the field of view of the optical system such as the camera, so that the camera can acquire a view of the tissue surface at the surgical space through the passage 130c of the first axial segment 132.

For example, the first axial segment 132, and thereby the passage 130c, may have an inner diameter of 6 mm to 12 mm (e.g., 8 mm to 10 mm, such as 9 mm), which may be substantially the same as the inner diameter of the proximal portion 122 and/or the outer diameter of the insertion tube 112 at the distal end 114 to ensure the best field of view of the camera.

In addition, the outer surface of the first axial segment 132 may have a diameter of 7 mm to 18 mm, for example, 7 mm to 14 mm (e.g., 10 mm to 13 mm, such as 12 mm). The first axial segment 132 may have a thickness of 0.5 mm to 2 mm. Instead of being cylindrical, the first axial segment 132 and/or the passage 130c defined thereby may have any other suitable shape moving axially, such as by gradually increasing in dimension in a constant (e.g., straight or frustoconical) or curved manner moving axially away from the proximal portion 122. Furthermore, the first axial segment 132 and/or the passage 130c may have a circular shape (as shown) or non-circular shape in cross-section (i.e., at a fixed axial position), such as ovular, squared or otherwise having straight segments, or other suitable shape.

The length L_short of the first axial segment 132 is that distance, measured in the axial direction, from the proximal portion 122 and/or the distal end 114 of the endoscope 110 to the aforementioned point of portion of the forward edge 130a of the cap 120 nearest thereto. The first axial segment 132 may also be referred to as the short segment.

The length L_short of the first axial segment 132 determines the length of the passage 130c, especially the extension length of the short side (or referred to as the biopsy channel side, shown in A in FIG. 6) of the forward edge 130a, thereby affecting the support of tissues such as the muscularis propria 100c by this first axial segment 132. The first axial segment 132 is too long, so that the overall extension length of the cap 120 is too long, which affects the maneuverability of the endoscope across angulated and tortuous anatomy 110. In addition, the farther the surgical space formed by the forward edge 130a is from the proximal portion 122, the farther the tissue is from the distal end 114 of the endoscope 110, and the extension length of the knife 116 needs to be increased in order to ensure that the knife 116 can engage the tissue, which will cause the maneuverability of the endoscope 110 to deteriorate. However, if the first axial segment 132 is too short, the closer the operation space formed by the forward edge 130a is from the proximal portion 122, the tissue bridging the operation space will easily block the field of view of the camera, thereby affecting the operation process.

In the embodiment of the disclosure, the length L_short of the first axial segment 132 is set between 2 mm and 15 mm. On the one hand, the short side of the first axial segment 132 can effectively support the tissue, so that the tissue is far away from the distal end 114 of the endoscope 110, increasing the field of view of the endoscope and increasing the operable space of the instrument. On the other hand, the operability of the knife 116 is also ensured. Exemplarily, the length L_short of the first axial segment 132 may be 4 mm to 10 mm (e.g., 5 mm). There may be markings on the forward edge 130a of the first axial segment 132 closest to the exit holes of the knife 116, which may be steep notches or printed markings for reference when the cap 120 is installed.

Exemplarily, the markings may be separate and steep notches which may span less than 2 mm, or 1 mm or less than 5, 3, 2 or 1 degree. Additionally, the markings may be notches forming alignment grooves 130b. The markings can ensure that after the endoscopic system is installed, the knife 116 is located on the opposite side of the extension of the second axial segment 134, so that the knife 116 can be exposed to the maximum extent in the lateral notch 136 mentioned herein below, which is convenient for surgical operation.

The forward edge 130a may extend a constant axial distance equal to the length L_short over the circumferential span S_short of the first axial segment 132 despite any separate and steep notch therein. Referring to FIG. 11, the circumferential span S_short can be defined by an angular measurement around the axis l. For example, the circumferential span S_short of the forward edge 130a may be 0 to 270 degrees, such as 0 to 180 degrees, 30 to 150 degrees (for example, 75 to 105 degrees) or 90 to 270 degrees (e.g., 130 to 220 degrees). Alternatively, the circumferential span S_short may be measured in a linear dimension, e.g., 0 mm to 10 mm (e.g., 4 mm to 8 mm). Alternatively, the axial length of the forward edge 130a may gradually increase as the forward edge 130a moves circumferentially from a starting point having a length L_short toward the second axial segment 134. The circumferential span S_short can also be referred to as the first circumferential span.

A second axial segment 134 of the distal portion 130 of the cap 120 protrudes forward relative to the first axial segment 132.

Here, the arrangement of the second axial segment 134 needs to be explained as follows:

As a first arrangement, the second axial segment 134 protrudes axially forward with respect to the first axial segment 132, in other words, the extending direction of the circumferential side wall of the second axial segment 134 is consistent with the extending direction of the axis l (referring to FIGS. 3a to 11), such that the distal portion 130 of the cap 120 extends axially forward from the proximal portion 122 to a forward edge 130a. It is understood that the extension directions of the proximal portion 122 and the distal portion 130 are parallel or substantially parallel to the axis l.

As a second arrangement, at least some of the circumferential side walls of the second axial segment 134 are inclined toward the axis l, so that at least some of the second axial segment 134 forms a conical or frustum-conical structure (hereinafter, the details will be described with reference to the figures).

Hereinafter, other structures of the endoscopic system provided by the present disclosure will be described in detail by taking the first arrangement of the second axial segment 134 as an example.

Referring to FIGS. 6 and 8, second axial segment 134 includes the portion or point of forward edge 130a that extends furthest from the proximal portion 122 and/or the distal end 114 as measured in the axial direction.

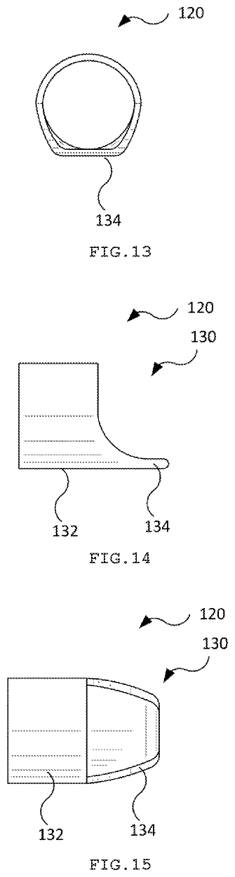

The second axial segment 134 of the cap 120 forms an extended side, for example, by extending circumferentially incompletely around the axis l, in other words, the second axial segment 134 extends circumferentially around the axis l by less than 360 degrees. The second axial segment 134 may be partially cylindrical, for example, having the same inner and/or outer radii as the first axial segment 132, or larger size. Alternatively, the second axial segment 134 may have another suitable shape. Furthermore, a part of the second axial segment 134 may have a circular (as shown in FIG. 10) or non-circular cross section (e.g., at fixed axial locations), e.g., a part of which has an oval, square shape or other shape with a straight segment, or other suitable shape (see, for example, FIGS. 13 to 15). Wherein, FIG. 13 is a second schematic structural view of the cap provided by an embodiment of the present disclosure, FIG. 14 is the left elevation view of FIG. 13, FIG. 15 is a top view of FIG. 13.

As shown in FIG. 9, the distal portion 130 of the cap 120 may be symmetric about a plane extending through the axis 114c of the insertion tube 112 and/or the cap 120, or may alternatively be asymmetric. Circumferential dimensions of the second axial segment 134 are discussed in further detail below.

The point or portion on the forward edge 130a of the second axial segment 134 having the length L_long (i.e., the maximum axial length of the forward edge 130a), which may be referred to as the elongated side, a first side, or a long side, is positioned across from that point or portion on the forward edge 130a of the first axial segment 132 having the length L_short (i.e., the minimum axial length of the forward edge 130a), which may be referred to as a second or short side. Wherein, the elongated side may be positioned radially opposite to the short side (e.g., with the axis l being positioned therebetween).

The length L_long of the second axial segment 134 is the distance measured in the axial direction from the forward edge 130a of the first axial segment 132 to the above-mentioned point or part of the forward edge 130a furthest from the first axial segment 132.

It can be understood that the length L_long of the second axial segment 134 determines the extension length of the long side B of the forward edge 130a, thereby affecting the degree of support to tissues such as the mucosal layer 100a. If the second axial segment 134 is too long, the structural strength of the second axial segment 134 will be greatly decreased, which will affect the support. At the same time, the length of the entire cap 120 will also become longer, which will affect the maneuverability of the endoscope across angulated and tortuous anatomy 110. In addition, the farther the surgical space formed by the forward edge 130a is from the proximal portion 122, the farther the tissue is from the distal end 114 of the endoscope 110, and the extension length of the knife 116 needs to be increased in order to ensure that the knife 116 can engage the tissue, which will cause the maneuverability of the endoscope 110 to deteriorate.

In the embodiment of the disclosure, the length L_long of the second axial segment 134 is set to 2 mm to 15 mm, so that, on the one hand, the second axial segment 134 can support longer (or more) mucosal layers 100a, increasing the field of view of the endoscope, thereby increasing the operable space of instruments such as the knife 116, on the other hand, it also ensures that the tissue will not be too far away from the distal end 114 to need extending the extension length of the knife 116, thereby ensuring the operability of the knife 116. In addition, the structural stability of the entire cap 120 and of the second axial segment 134 is also ensured.

Exemplarily, the length L_long of the second axial segment 134 may be 3 mm to 10 mm (e.g., approximately 3 mm to 5 mm) or 7 mm to 15 mm (e.g., approximately 9 mm to 11 mm). Wherein, the length L_long of the second axial segment 134 is preferably no more than 10 mm, more preferably 2 mm to 5 mm.

Instead of or in addition to being defined in absolute terms, the length L_distal of the distal portion 130 and/or the length L_long of the second axial segment 134 may be defined with respect to the size or dimensions of the cap 120 and/or another portion of the endoscope 110. For example, the length L_long of the second axial segment 134 may be between 0.25 and 1.5 times the diameter of the outer surface of the passage 130c and/or the first axial segment 132, or may be between 0.25 and 1.5 times the other lateral dimensions of the passage 130c and/or the first axial segment 132 (e.g., 0.25 to 0.75 times (such as 0.4 to 0.6 times) or 0.75 to 1.5 times (such as 0.9 to 1.1 times)).

Referring to FIG. 11, the forward edge 130a may extend a constant axial distance of length L_long over the circumferential span S_long of second axial segment 134. The circumferential span S_length may be defined by an angular measurement around the axis l of the distal portion 130.

It should be noted that the larger the circumferential span S_length of the forward edge 130a, the stronger the radial support force of the forward edge 130a to the tissue, the better the radial support effect on the tissue such as the mucosal layer 100a, ensuring that the tissues on both sides of the distal portion 130 exit the middle passage 130c in a better way, thereby increasing the field of view of the endoscope and increasing the operable space of instruments such as the knife 116. However, if the circumferential span S_long of the forward edge 130a is too long, the tissue is too far away from the distal end 114 of the endoscope 110, thereby necessitating an extended extension of the knife 116, thereby affecting the maneuverability of the knife 116.

In the embodiment of the disclosure, the circumferential span S_length of the forward edge 130a is set to 0 to 180 degrees, so as to improve the radial support effect on tissues such as the mucosal layer 100a and ensure that the tissues on both sides will not affect the field of view of the endoscope. At the same time, making the tissue such as the submucosa 100b moderate from the distal end 114 ensures the maneuverability of the knife 116. Exemplarily, the circumferential span S_length of the forward edge 130a may be 45 to 180 degrees (e.g., 60 to 120 degrees, such as 75 to 105 degrees) or 5 to 90 degrees (e.g., 5 to 45 degrees, such as 5 to 20 degrees).

In some examples, the forward edge 130a may gradually decrease in axial length from a singular point having the length L_long moving peripherally therealong toward the first axial segment 132. The peripheral span S_long may also be referred to as the second peripheral span.

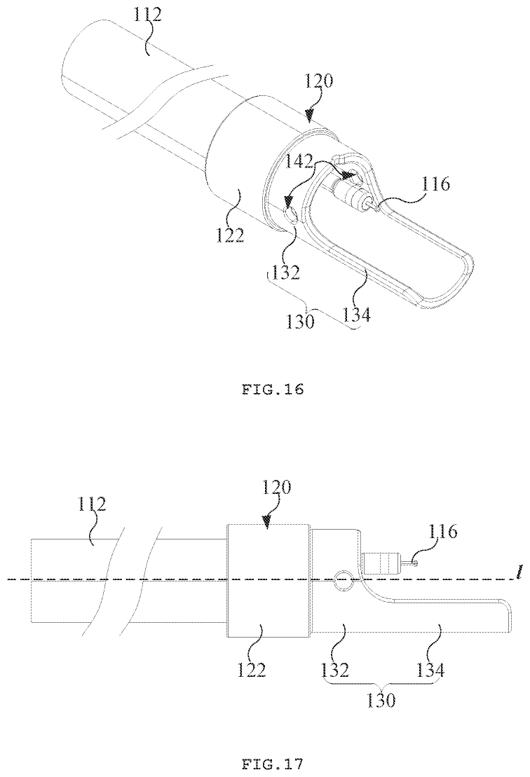

FIG. 16 is a third schematic structural view of the endoscopic system provided by an embodiment of the present disclosure, and FIG. 17 is a left elevation view of the endoscopic system in FIG. 16. Referring to FIGS. 16 and 17, an alternative embodiment of the cap 120 is shown in FIGS. 16 and 17, both of which show the length L_long of the second axial segment 134 of the cap 120 is longer than the length L_long shown in FIGS. 3a-12.

FIGS. 16 and 17 also show that the circumferential span S_length of the second axial segment 134 of the cap 120 is smaller than the circumferential span S_length shown in FIGS. 3a to 12. Yet another embodiment of the cap 120 is shown in FIGS. 13-16, wherein the distal portion 130 has a non-circular cross-sectional shape. More specifically, the second axial segment 134 transitions axially from the circular cross-sectional shape of the first axial segment 132 to have a flat side (e.g., the bottom side as shown in FIGS. 13 and 14), such as planar as shown.

The forward edge 130a may gradually vary in length moving circumferentially therearound, for example, to avoid sharp outside corners that might otherwise poke, catch, or abrade tissue. For example, as shown, the second axial segment 134 may include rounded corners that transition moving circumferentially from the maximum axial length L_long.

As referenced above, the second axial segment 134 of the cap 120 forms the extended side, for example, by not extending circumferentially entirely around the passage 130c and/or the axis 114c. Rather, over the axial length L_long of the second axial segment 134, the second axial segment 134 may extend circumferentially around the axis l varying amounts to the forward edge 130a, for example, gradually decreasing in circumferential distance around the axis l moving axially away from the first axial segment 132 (e.g., away from the distal end 114 of the insertion tube 112).

For example, over the length L_long of the second axial segment 134, the second axial segment 134 may extend circumferentially around the axis l on average less than 180 degrees (e.g., an average of less than 135 degrees). Instead of or additionally, over a majority (e.g., greater than 50%, 55%, 60%, 70%, or more) of the length L_long of the second axial segment 134, the second axial segment 134 may extend circumferentially around the axis 114c 180 degrees or less.

Referring to FIGS. 6 and 8, the forward edge 130a of the distal portion 130 may be concave, for example, the forward edge 130a may extend forward a different distance to define a lateral notch 136, the lateral notch 136 extends laterally through the passage 130c (e.g., perpendicular to the axis l of the cap 120), and extends across the entire forward edge 130a. It will be appreciated that the lateral notch 136 forms the surgical volume described above. Referring to FIGS. 8 and 9, in use, the knife 116 may insert into the lateral notch 136 to cut tissue.

In use, tissue is joined by forward edge 130a on the short side (i.e., has a length L_short) and the long side (i.e., has a length L_long) and is suspended across forward edge 130a. In order to ensure that the knife 116 effectively cuts the tissue overhanging the forward edge 130a, the tip of the knife 116 can be axially flush or protrude from the lateral notch 136, for example, the knife 116 extends forward axially into and/or beyond the lateral notch 136 to engage and cut tissue. It should be noted that the lateral notch 136 corresponds to the longitudinal position (e.g., the vertical position of FIGS. 8 and 9) of the knife 116 (e.g., the outlet 114b of the distal end 114 of the insertion tube 112).

Referring to FIG. 6, exemplarily, the forward edge 130a is concave between the first axial segment 132 and the second axial segment 134 to utilize the transition structure between the first axial segment 132 and the second axial segment 134, so as to avoid forming a concave structure on the first axial segment 132 or the second axial segment 134 alone, which will affect the structural strength of the first axial segment 132 and the second axial segment 134.

Referring to FIG. 8, the forward edge 130a is axially recessed relative to a plane 140 to form above-mentioned lateral notch 136, the plane 140 extends across the passage 130c (for example, across the axis l) and intersects at the forward edge 130a on the first axial segment 132 (i.e. the short side with length L_short) and the second axial segment 134, in other words, the plane 140 intersects with both the short side of the forward edge 130a and the portion on the second axial segment 134, respectively. Lateral notch 136 does not pass through cap 120 (as indicated by plane 140) or where the length is L_long (as indicated by plane 140′).

Wherein, two opposite sides of the plane 140′ at the distal end portion 130 intersects with the forward edge 130a, in other words, the plane 140 is specifically a plane 140′ in one example, and the plane 140′ is specifically intersects with the short side and the long side of the forward edge 130a, and the forward edge 130a is axially recessed relative to the plane 140′ to form the above-mentioned lateral notch 136, that is, the lateral notch 136 is located on the axial rear side of the plane 140′ (referring to FIG. 8).

In the case of the forward edge 130a is axially recessed relative to plane 140 and/or plane 140′, the forward edge 130a may be considered to define lateral notch 136. The lateral notch is the axially forward region of the forward edge 130a and the axially rearward region of the planes 140, 140′. The cap 120 may be coupled to insertion tube 112 in a direction that knife 116 may extend into and/or through lateral notch 136.

It should be noted that the forward edge 130a is axially recessed relative to the plane 140 and/or the plane 140′ to form a lateral notch 136, so that when tissue such as the submucosa 100b is suspended on the surgical space formed at the forward edge 130a, it can sink into the lateral notch 136, so that the submucosa 100b is closer to the distal end 114 of the endoscope 110, so that the submucosa 110b can be effectively cut while ensuring that the instrument such as the knife 116 extends a shorter length, thereby improving the operability of the knife 116.

It will be appreciated that depending on the softness of the tissue, the extent to which it sinks into the lateral notch 136 when suspended on the forward edge 130 will change; for example, softer tissue may sink into lateral notch 136 to a greater extent; the harder the tissue is, the smaller the degree of its depression to the lateral notch 136 will be, therefore, during the operation, the knife 116 can be adjusted in the axial length in time according to the actual situation.

Referring to FIG. 6, in some examples, when the extending direction of the circumferential sidewall of the second axial segment 134 coincides with the extending direction of the axis l, the lateral notch 136 on the forward edge 130a is located between the short side A of the forward edge 130a and the axis l, so as to avoid the excessive depression of the lateral notch 136, and reduce the transition between the first axial segment 132 and the second axial segment 134, thereby ensuring the radial support of the second axial segment 134.

The distal portion 130 is preferably formed of a transparent material, such as polyvinylchloride (PVC), polyethylene, styrene, polycarbonate, acrylic, thermoplastic elastomers, or other transparent and/or colorless materials. In some embodiments, the material forming the distal portion 130 is elastically deformable, such that distal portion 130 may elastically deflect or deform under higher loading events when coupled to the endoscope 110 and/or inserted into a patient. The distal portion 130 is preferably formed as a singular (e.g., unitary), monolithic component. The distal portion 130 may further be formed with the proximal portion 122 as a singular (e.g., unitary), monolithic component with the distal portion 130 and the proximal portion 122 being formed of the same material during the same operation. Alternatively, proximal portion 122 may be formed as a separate component and/or of a different material and coupled to the distal portion 130.

The second arrangement of the second axial segment 134 will be described in detail below with reference to the accompanying drawings.

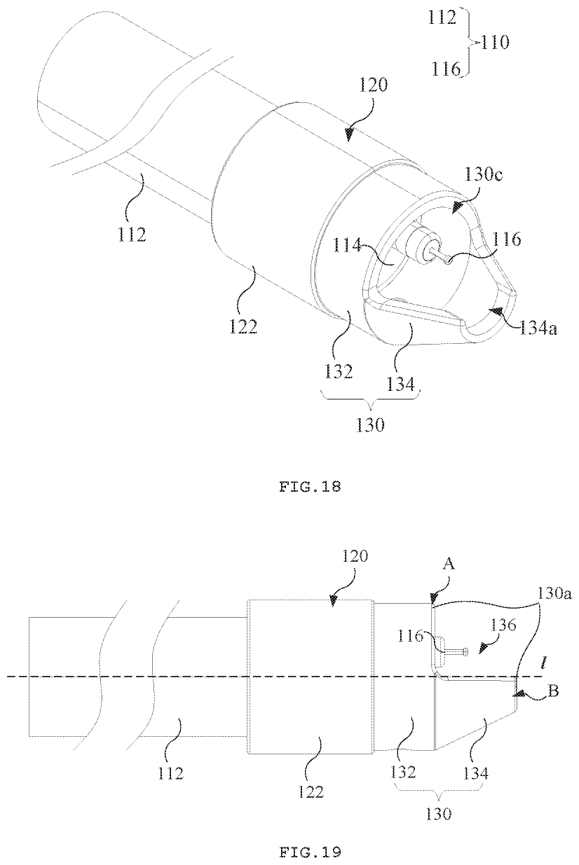

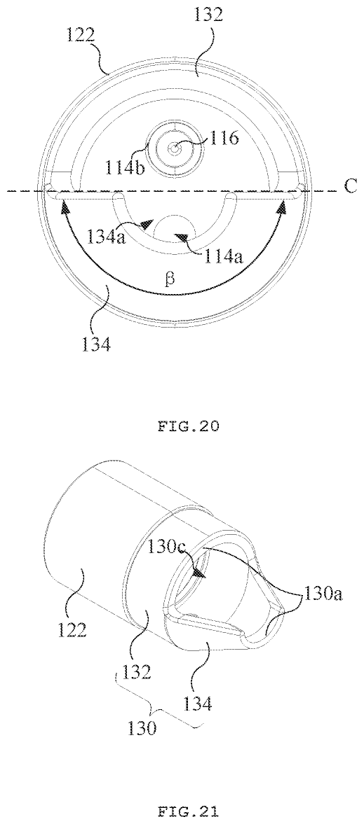

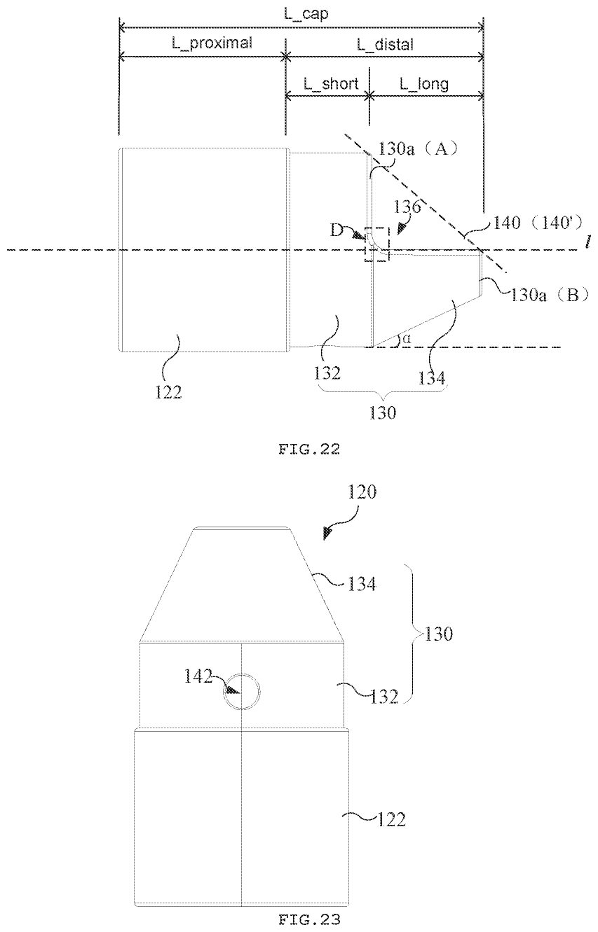

FIG. 18 is a fourth schematic structural view of the endoscopic system provided by an embodiment of the present disclosure, FIG. 19 is a left elevation view of FIG. 18, FIG. 20 is a front view of FIG. 18, FIG. 21 is a schematic structural view of the cap in FIG. 18, FIG. 22 is the left elevation view of FIG. 21, and FIG. 23 is a bottom view of FIG. 21.

Referring to FIGS. 18 to 23, the difference from the above-mentioned first arrangement of the second axial segment 134 is that in the second arrangement, in the second axial segment 134 of an embodiment of the disclosure, the circumferential sidewall of at least some segments is inclined toward the axis l in a direction away from the first axial segment 132, and one end of at least some segments is located at the end of the second axial segment 134 away from the first axial segment 132, in other words, over at least part of the second axial length, the second axial segment 134 is inclined toward the axis l in a direction away from the first axial segment 132. Wherein, one end of the inclined portion of the second axial segment 134 extends to the second axial length of the second axial segment 134 (i.e., at the long side B of the forward edge 130a).

In some examples, a part of the long side B of the second axial segment 134 close to the forward edge 130a is inclined toward the axis l in a direction away from the first axial segment 132, so that a part of the second axial segment 134 that is close to the foremost end forms a conical-like structure.

In some other examples (as shown in FIGS. 18 to 23), the second axial segment 134 gradually inclines from the first axial segment 132 to the axis l throughout the second axial length, so that the entire second axial segment 134 forms a cone-like structure.

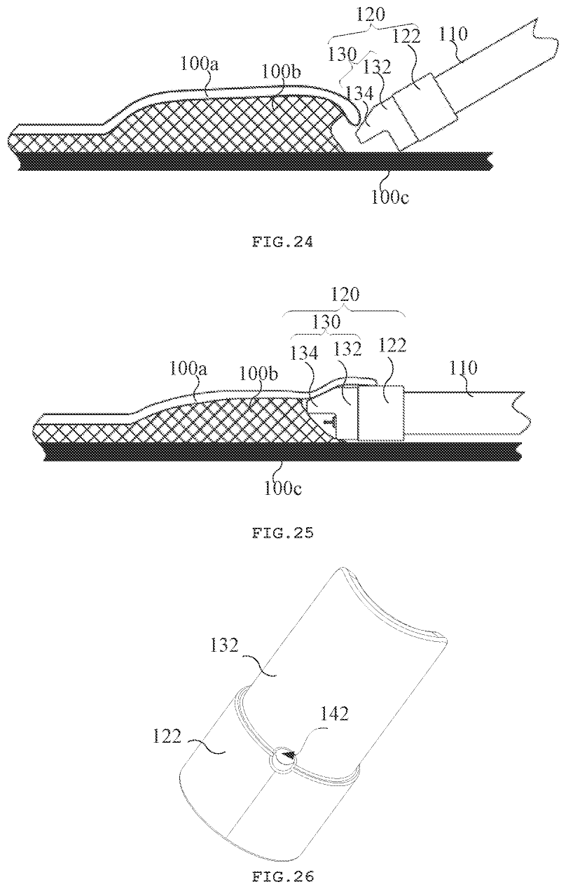

FIG. 24 is a first schematic structural view of the endoscopic system in FIG. 18 performing gastrointestinal surgery, and FIG. 25 is a second schematic structural view of the endoscopic system in FIG. 18 performing gastrointestinal surgery. Referring to FIGS. 24 and 25, when a gastrointestinal endoscopic surgery (such as POEM or ESD) is performed, after the mucosal layer 100a is cut through by the knife of the endoscopic system of the embodiment of the disclosure, the front end of the second axial segment 134, which resembles a conical structure, can be similar to a shovel, which can efficiently and effectively lift the mucosal layer 100a, so that the entire second axial segment 134 gradually enters the submucosa 100b between the mucosal layer 100a and the muscularis propria 100c, and during the endoscope carrying cap 120 continues to extend into the submucosa 100b, the mucosal layer 100a is supported in the inclined outer surface of the second axial segment 134 and the flat outer surface of the first axial segment 132, the muscularis propria 100c is supported on one outer surface on the short side A of the forward edge 130a, and the submucosa 100b is suspended in traction between the forward edge 130a of the first axial segment 132 and the second axial segment 134, thereby increasing the field of view of the endoscope and increasing the operating space at the front end of the second axial segment 134.

In addition, in the ESD operation, the second axial segment 134 disposed obliquely scoops up the mucosal layer 100a effectively and quickly after the knife cuts through the mucosal layer 100a. At the same time, the mucosal layer 100a is supported on the outer surface of the second axial segment 134 during the endoscope 110 continuously extends into the submucosa layer 100b, so as to facilitate the effective and rapid peeling off of the mucosal layer 100a from the muscularis propria 100c. It can be understood that the structure disposed obliquely of the second axial segment 134 is more suitable for the pocket or tunnel methods of ESD surgery.

Based on the above, it can be seen that the greater the angle β (which can be understood as the second circumferential span of the forward edge 130a) extending about the axis l, the larger the radial support force of the second axial segment 134 to the tissue is, the better the radial support effect on tissues such as mucosal layer 100a, so as to ensure that the tissues on both sides of the distal portion 130 will leave the middle passage 130c in a better way, thereby increasing the field of view of the endoscope and increasing the operable space of instruments such as knife 116.

However, if the angle β extended by the second axial segment 134 about the axis l is too large, the tissue will be too far away from the distal end 114 of the endoscope 110, thereby necessitating an extended extension of the knife 116, thereby affecting the maneuverability of the knife 116.

Referring to FIGS. 19 and 20, in this example, the longitudinal section passing through the axis l is the first section C, and the second axial segment 134 and the short side A of the forward edge 130a are located on both sides of the first section C, respectively. In other words, the entire circumferential sidewall of the second axial segment 134 does not exceed the first section C, for example, the second axial segment 134 extends about the axis at an angle β of 0 to 180 degrees, so as to make the distance between the tissue such as the submucosa layer 100b and the distal end 114 moderate to ensure the operability of the knife 116, while improving the radial support effect on tissues such as the mucosal layer 100a, thereby ensuring that the tissues on both sides will not affect the field of view of the endoscope.

Referring to FIG. 20, exemplarily, the two opposite peripheral edges of the second axial segment 134 along the circumferential direction is located on the first section C, that is, the second axial segment 134 extends 180° about the axis l, so as to ensure that the field of view of the endoscope and the maneuverability of the knife 116 simultaneously.

In this example, the lowest position of the lateral notch 136 on the forward edge 130a is flush with or below the first section C. Based on the above, it can be seen that the deeper the lateral notch 136 is, the more the tissue such as the submucosa 100b can sink into the lateral notch 136 when it is suspended on the surgical space formed by the forward edge 130a, so that the submucosa 100b is closer to the distal end 114 of the endoscope 110, and the instrument, such as the knife 116, can also effectively cut the submucosa 100b while ensuring that it protrudes out a relatively short length, thereby improving the maneuverability of the knife 116.

In addition, the deeper the lateral notch 136 is, the larger the surgical space formed between the forward edges 130a is, the larger the field of view of the endoscope is, and the larger the surgical operation space is.

Whereas in contrast to the first arrangement, in the second arrangement, since the second axial segment 134 is arranged as a conical-like structure inclined toward the axis l, the radial support thereof is stronger. Based on this, the degree of depression of the lateral notch 136 can be deeper than that of the first arrangement without affecting the radial support of the second axial segment 134.

Referring to FIG. 22, in some examples, the forward edge 130a at the lateral notch 136 is configured as an arc transition from the first axial segment 132 to the second axial segment 134 (see D in FIG. 22), so as to improve the structural strength of the forward edge 130a at the transverse notch 136 and ensure the radial support of the second axial segment 134.

In some examples, in order to prevent the tissue attached to the second axial segment 134, such as the mucosal layer 100a, from blocking the optical path of the optical system such as a camera, the circumferential sidewall of the second axial segment 134 is arranged in a staggered manner with the optical system of the distal end 114 of the insertion tube in the projection of the distal end 114 of the insertion tube, so that the circumferential side wall of the second axial segment 134 is staggered and a block of the optical path of the camera may be prevented, so as to ensure that the camera can capture the images of the forward edge 130a and the tissue at the front end of the endoscope 110 in a better way, thereby ensuring the effective process of the operation.

For example, an avoidance port 134a may be formed at an end of the second axial segment 134 away from the first axial segment 132, and the axial projection of the optical system on the second axial segment 134 is located in the avoidance port 134a.

It is appreciated that the formation of the avoidance port 134a is correlative to the second axial length of the second axial segment 134 and the inclination angle α (refer to FIG. 8). When the inclination angle α of the second axial segment 134 is constant, by controlling the second axial length, the portion of the forward edge 130a of the front end of the second axial segment 134 (that is, the end away from the first axial segment 132) does not reach axis l, so that an avoidance port 134a is created at the front end of the second axial segment 134 to increase the field of view of the endoscope.

Of course, in other examples, the inclination angle of the second axial segment 134 can be adjusted to ensure that the tissues attached to the second axial segment 134 will not have too much influence on the optical path of the camera while the second axial segment 134 effectively lifts the mucosal layer 100a and enters the submucosa layer 100b, so as to ensure the field of view of the endoscope.

In addition, if the angle α is too large, the second axial segment 134 will get too close to the axis l, in this way, after the stretched tissue such as the mucosal layer 100a is attached to the outer wall, it will block the front of the camera in the endoscope, which is negative to the field of view of the endoscope. In addition, when the second axial length is constant, the larger the angle α is, the farther the tissue such as the submucosa is from the lateral notch 136, resulting in a more difficult operation of the knife.

Exemplarily, the angle α at which the circumferential sidewall of the second axial segment 134 is inclined to the axis is 15°-60°, so that it is easier to lift the mucosal layer 100a in the second axial segment 134 and drill into the mucosal layer 100a and the muscularis propria 100c, and when the endoscope continues to penetrate into the submucosa 100b, it can lift and support the mucosal layer 100a in a better way, thereby opening up the operation space and making the operation more maneuverable. In addition, it can also improve or avoid the blocking of the optical path of the camera by the tissue attached to the outer surface of the second axial segment 134, such as the mucosal layer 100a, so as to ensure the field of view of the endoscope.

For example, the inclination angle α of the second axial segment 134 may be a suitable angle value, such as 15°, 30°, 45° or 60°, etc.

It is appreciated that the angle β extending about the axis l in the second axial segment 134 and the inclination angle α jointly determine the maneuverability of the endoscope. For example, when β is small, the inclination angle α can be appropriately increased, so that the mucosal layer 100a can be effectively and quickly scooped up and supported on the second axial segment 134 to ensure the endoscopic view, while ensuring that the tissue is not far away from the distal end 114. For example, when the angle α at which the circumferential sidewall of the second axial segment 134 is inclined to the axis is 60°, the extension angle β of the second axial segment 134 may be 180°.

In some other examples, the end of the second axial segment 134 away from the first axial segment 132 coincides with the axis l, that is, the second axial segment 134 forms a conical structure. For example, when the inclination angle α is constant, the second axial length L_long can be extended so that the long side of the forward edge 130a completely coincides with the axis l.

Alternatively, when the second axial length L_long is constant, the inclination angle α is increased so that the long side B of the forward edge 130a meets with the axis l, so that the second axial segment 134 is formed into a conical structure, so that the cap 120 may drill into between the mucosal layer 100a and the muscularis propria 100c more easily, and it is very easy to lift the mucosal layer 100a, which is more effective for expanding the surgical operation space and peeling off the mucosal layer 100a.

Additionally, the second axial length L_long may, in conjunction with the inclination angle α, affect the maneuverability of an instrument, such as the knife 116. In the disclosure, when the second axial length is small, the inclination angle α can be appropriately increased, so as to ensure that the tissue suspended on the forward edge 130a, such as the submucosa 100b, is not too far away from the lateral notch 136, while the cap 120 can more easily drill into between the mucosal layer 100a and the muscularis propria 100c and lift the mucosal layer 100a very easily, so that the knife 116 can engage and cut the tissue even if it protrudes out by an appropriate length, ensuring the maneuverability of the knife.

Exemplarily, the second axial length L_long may be 2 mm-15 mm, for example, the second axial length L_long may be 2 mm, 4 mm, 8 mm or 15 mm and other suitable length values. In an actual setting, the second axial length L_long may be 4 mm, and the angle α at which the circumferential sidewall of the second axial segment 134 is inclined to the axis may be 60° or greater than 60°.

It should be noted that due to the existence of the first axial segment 132, the axial distance between the mucosal layer 100a on the second axial segment 134 and the camera is extended, so that the backward part of the mucosal layer 100a is first supported on the first axial segment 132, so that there is a transitional support for the mucosal layer 100a located in the second axial segment 134. Compared with a situation where the entire distal section 130 is inclined from the front end of the proximal section 122, an occlusion of the front of the camera by the mucosal layer 100a over the second axial segment 134 can be improved to ensure sufficient operating space.

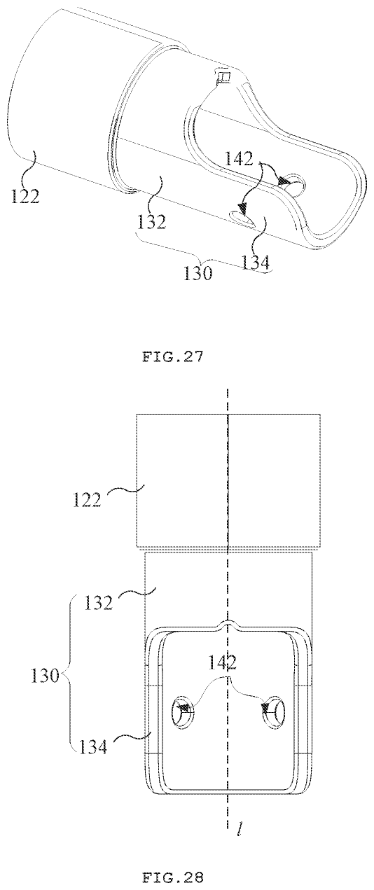

FIG. 26 is a fifth schematic structural view of the cap provided by an embodiment of the present disclosure, FIG. 27 is a sixth schematic structural view of the cap provided by an embodiment of the present disclosure, and FIG. 28 is a top view of FIG. 27. Referring to FIGS. 5, 16, 23 and 26-28, the cap 120 may also include one or more drain holes 142 configured as an outlet for water or air trapped by the cap 120, for example in the passage 130c.

As shown in FIGS. 16 and 23, for example, at least one drain hole 142 is disposed in the first axial segment 132 at a distance from the proximal end portion 122 of the endoscopic attachment and/or the distal end 114 of the endoscope less than the length L_short of the first axial segment 132 of the distal end 114 of the cap 120.

Referring to FIG. 26, as another example, at least one drain hole 142 may also be partially disposed in the first axial segment 132, for example, disposed at a junction between the first axial segment 132 and the proximal portion 122.

As a further example, at least one drain hole 142 may be provided in the second axial segment 134.

As shown in FIG. 16, the cap 120 may include a plurality of drain holes 142. The plurality of drain holes 142 have a common axial position, for example, the cap 120 may include two drain holes 142, the two drain holes 142 are located at the same axial position, and the connecting line between the two drain holes 142 may pass through the passage 130c; for example, two opposing drain holes 142 are spaced 180 degrees apart from each other.

Referring to FIGS. 27 and 28, in addition, in the case of two drain holes 142, the drain holes 142 may be located on two opposite sides of the elongated side perpendicular to the axial direction, and/or a point or portion thereof whose length is L_long. For example, the cap 120 may include one or more drain holes 142 in the extension side (e.g., in second axial segment 134) and/or circumferentially aligned with the extension side.

Additionally, referring to FIG. 7, by locating the drain hole 142 away from the transition region between the first axial segment 132 and the second axial segment 134, it is helpful to reduce stress concentrations and potential failure regions. For example, when the cap 120 is installed on the endoscope 110, the cap 120 is prevented from tearing from the forward edge to the drain hole 142. The diameter of the drain hole 142 may be, for example, 0.5 mm to 4 mm (e.g., about 3 mm).

Referring to FIGS. 12, 24 and 25, an endoscope 110 with a cap 120 is illustrated in use during POEM procedures. The distal end 114 of the endoscope 110 (including the cap 120 coupled thereto) is inserted in the submucosa 100b between the mucosal layer 100a and the muscularis propria 100c. The forward edge 130a of the cap 120 engages tissue on the first and second axial segments 132, 134 and suspends tissue of submucosa 100b therethrough. The mucosal layer 100a detached from the muscularis propria 100c is supported on the outer surface of the second axial segment 134, that is, the outer surface of the long side of the forward edge 130a, the muscularis propria 100c which is detached from the mucosal layer 100a and has the submucosa 100b is supported on the outer surface of the short side of the forward edge 130a, and the tissue of the submucosa 100b extending between the first axial segment 132 and the second axial segment 134 can be maintained therebetween under tension and/or may protrude into lateral notch 136. As shown in FIGS. 12 and 25, the extended side of the cap 120 may be disposed between the mucosal layer 100a and the knife 116, or alternatively, may be disposed between the muscularis propria 100c and the knife 116.

The knife 116 is then extended to engage the submucosa 100b and manipulated to cut the submucosa 100b, e.g., the tip of the knife 116 is pulled through the submucosa 100b for cutting the tissue thereof. The endoscope 110 is then further inserted between the mucosal layer 100a and the muscularis propria 100c to suspend the various tissues of the submucosa 100b passing therethrough, and the knife 116 is again extended to engage the submucosa 100b and manipulated to cut the submucosa 100b.

Between cutting of tissue and further insertion of endoscope 110, the knife 116 may be retracted (e.g., out of lateral notch 136) behind the forward edge 130a. The process of cutting and further insertion of the endoscope 110 is repeated until the lower esophageal sphincter 104 is reached, at which point a myotomy (i.e., incision) of the musculature of the lower esophageal sphincter 104 is performed.

During the procedure, a fluid solution may be injected through either or both of the nozzle 114a or the tip of the knife 116, which may serve to separate tissue, remove any loose tissue, and clear or prevent smoke (which might otherwise obstruct the field of view through the endoscope 110) and realize an effect of “underwater” imaging.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. An endoscopic attachment, comprising:
a proximal portion configured to be coupled to an insertion tube of the endoscope; and
a distal portion coupled to the proximal portion and extending forwardly from the proximal portion to a forward edge;
wherein the distal portion comprises:
a first axial segment that extends from the proximal portion axially to the forward edge by a first axial length, and defines a passage, wherein the first axial segment extends circumferentially entirely around an axis of the passage; and
a second axial segment that extends a second axial length from the first axial segment to the forward edge, and over at least part of the second axial length, the second axial segment extends circumferentially around the axis of the proximal portion less than 360 degrees.

2. The endoscopic attachment according to claim 1, wherein over the second axial length, the second axial segment extends circumferentially about the axis less than or equal to 180 degrees on average.

3. The endoscopic attachment according to claim 1, wherein a lateral notch which transverses to said axis and extends through the passage is formed over the forward edge.

4. The endoscopic attachment according to claim 3, wherein an open surgical space is enclosed between the forward edges, and the surgical space is configured to accommodate a knife extending from the distal end of the insertion tube to the distal portion;
wherein the tip of the knife is axially flush with or protruding from the lateral notch.

5. The endoscopic attachment of claim 3, wherein the forward edge is axially recessed to form the lateral notch with respect to a plane extending laterally across the passage and intersecting both a short side of the forward edge and the second axial segment, respectively, the short side being the portion of the forward edge at a first axial length in the first axial segment.

6. The endoscopic attachment of claim 5, wherein the plane intersects the long side of the forward edge, the long side being the portion of the forward edge at the second axial length in the second axial segment.

7. The endoscopic attachment of claim 1, wherein the forward edge extends the first axial length over a first circumferential span of 0 to 180 degrees about the axis.

8. The endoscopic attachment of claim 7, wherein the forward edge extends the first axial length over a first circumferential span of 30 to 150 degrees about the axis.

9. The endoscopic attachment of claim 1, wherein the forward edge extends the second axial length over a second circumferential span of 0 degrees to 180 degrees about the axis.

10. The endoscopic attachment of claim 1, wherein the first axial length is 2 to 15 mm.

11. The endoscopic attachment of claim 1, wherein the second axial length is 2 to 15 mm.

12. The endoscopic attachment of claim 11, wherein the second axial length is 2 to 5 mm.

13. The endoscopic attachment of claim 1, wherein the second axial segment transitions moving axially from the circular cross-section shape of the first axial segment to having a flattened side.

14. The endoscopic attachment of claim 1, further comprising one or more drain holes.

15. The endoscopic attachment of claim 14, wherein at least one of the drain holes is located in the first axial segment, and a distance thereof from the proximal portion is smaller than the first axial length;
alternatively, at least one of the drain holes is located at a junction of the proximal portion and the first axial segment;
alternatively, at least one of the drain holes is located on the second axial segment;
alternatively, at least two of the drain holes are located on the second axial segment, and the at least two of the drain holes are circumferentially aligned by extension sides formed by the second axial segment.

16. The endoscopic attachment of claim 1, wherein an adhesive tape with a non-adhesive removable coating is secured to a rearward edge, and during mounting on the insertion tube, the adhesive tape is folded backward over the rearward edge, wherein the rearward edge is an edge of an end of the proximal portion away from the distal portion.

17. The endoscopic attachment of claim 16, wherein the adhesive tape is secured to a surface of the insertion tube by peeling off the non-adhesive coating.

18. The endoscopic attachment of claim 1, wherein a circumferential sidewall of the second axial segment extends in a direction coincident with the axis, such that the distal portion extends axially forward from the proximal portion to the forward edge.

19. The endoscopic attachment of claim 18, wherein the lateral notch over the forward edge is provided between the short side of the forward edge and the axis; the short side is a portion of the forward edge at a first axial length in the first axial segment.

20. The endoscopic attachment of claim 1, wherein at least a portion of said second axial segment is inclined toward said axis in a direction away from said first axial segment, and an end of the at least portion of the second axial segment is located at the second axial length of the second axial segment.

21. The endoscopic attachment of claim 20, wherein the second axial segment is gradually inclined from said first axial segment towards said axis throughout the second axial length.

22. The endoscopic attachment of claim 20, wherein the second axial segment and the short side of the forward edge are located on two different sides of a first section plane, respectively; wherein the first section plane is a longitudinal section passing through the axis, and the short side is the portion of the forward edge at the first axial length in the first axial segment.

23. The endoscopic attachment of claim 22, wherein circumferentially opposite side edges of the second axial segment are located on the first section plane.

24. The endoscopic attachment of claim 22, wherein the lowest portion of the lateral notch over the forward edge is flush with or below the first section plane.

25. The endoscopic attachment according to claim 20, wherein a projection of the circumferential sidewall of the second axial segment on the distal end of the insertion tube is arranged in a staggered manner with respect to the optical system at the distal end of the insertion tube.

26. The endoscopic attachment according to claim 25, wherein an avoidance port is formed at an end of the second axial segment away from the first axial segment, and an axial projection of the optical system on the second axial segment is located in the avoidance port.

27. The endoscopic attachment of claim 20, wherein an end of the second axial segment away from the first axial segment meets with the axis.

28. The endoscopic attachment according to claim 20, wherein the circumferential sidewall of the second axial segment is inclined toward the axis with an angle of 15°-60°.

29. An endoscopic system comprising:
an endoscope having an insertion tube and a knife extendable from the distal end of the insertion tube; and
an endoscopic attachment, the endoscopic attachment comprising:
a proximal portion coupled to the insertion tube; and
a distal portion coupled to the proximal portion and extending forwardly from the proximal portion to a forward edge:
wherein the distal portion comprises:
a first axial segment extending from the proximal portion axially to the forward edge by a first axial length, and defining a passage, the first axial segment extending circumferentially entirely around an axis of the passage;
a second axial segment extending a second axial length from the first axial segment to the forward edge, wherein over at least part of the second axial length, the second axial segment extends circumferentially around said axis of the proximal portion less than 360 degrees.

30. The endoscopic system of claim 29, wherein the axis is located radially between the knife and a portion of the second axial segment extending the second axial length to the forward edge.

31. A cap for an endoscope comprising:
a coupling portion configured to be coupled to an insertion tube of an endoscope having a knife extendable from the insertion tube; and
a shroud portion extending axially from the coupling portion to a forward edge, the shroud portion defining a passage having an axis and a lateral notch extending through the channel transverse to the axis.

32. The cap for an endoscope according to claim 31, wherein said lateral notch is an axially forward region of the forward edge and an axially rearward region of a plane which extends through the passage and intersects the forward edge on the opposite sides of the forward edge.

33. The cap for an endoscope according to claim 32, wherein the forward edge is recessed with respect to the plane.

34. The cap for an endoscope according to claim 31, wherein the coupling portion is configured to be coupled to the insertion tube in a direction such that the knife may extend into the lateral notch.

35. The cap for an endoscope according to claim 31, wherein the shroud portion extends circumferentially about said axis less than or equal to 180 degrees on average over an axial length including the forward edge.
